(19) United States
(12) Patent Application Publication
(10) Pub. No.: US 2013/0324748 A1
(43) Pub. Date: Dec. 5, 2013

(54) PROCESS FOR PREPARATION OF LEVONORGESTREL

(71) Applicants: Swapnil Ajit Zadbuke, Pune (IN); Kishor Gulabrao Mehare, Pune (IN); Himanshu Madhav Godbole, Pune (IN); Girij Pal Singh, Pune (IN)

(72) Inventors: Swapnil Ajit Zadbuke, Pune (IN); Kishor Gulabrao Mehare, Pune (IN); Himanshu Madhav Godbole, Pune (IN); Girij Pal Singh, Pune (IN)

(73) Assignee: LUPIN LIMITED, Mumbai, Maharashtra (IN)

(21) Appl. No.: 14/000,056

(22) PCT Filed: Feb. 14, 2012
(86) PCT No.: PCT/IB2012/050658

§ 371 Date: Aug. 16, 2013

(30) Foreign Application Priority Data
Feb. 17, 2011 (IN) 198/KOL/2011

(51) Int. Cl. C07J 1/00

(52) U.S. Cl. CPC C07J 1/00
USPC 552/648

(57) ABSTRACT

The present invention provides an improved process for preparation of levonorgestrel (3) which comprises of hydrolysis of 13β-ethyl-3-methoxy-17α-ethynyl-gona-2,5(10)-dien-17β-ol (2) with an acid in aprotic solvent. The present invention also provides a novel process for purification of crude levonorgestrel (3) by recrystallization from N,N-dimethyl formamide-water; methanol-water mixture.

### TECHNICAL FIELD OF INVENTION

The present invention is related to an improved process for preparation of levonorgestrel (3) and a novel process for purification of crude levonorgestrel (3).

### BACKGROUND OF THE INVENTION

Levonorgestrel is a synthetic progestational and ovulation inhibiting steroid used as an active ingredient in some oral contraceptives as well as contraceptive implants. Levonorgestrel is chemically known as 13β-ethyl-17α-ethynyl-17β-hydroxygon-4-en-3-one and is represented by following structure.

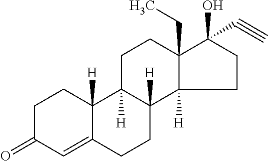

Levonorgestrel is most commonly used in combination with ethinyl estradiol. The combination of levonorgestrel with ethinyl estradiol is sold under the trade name Levora® by Watson labs in USA. Levonorgestrel was generically and specifically disclosed in U.S. Pat. No. 3,959,322, which is expired.

Very few references are directed towards synthesis of levonorgestrel (3), most relevant to the present invention are discussed herein below:

The product patent for levonorgestrel (3), U.S. Pat. No. 3,959,322 teaches a process for preparation of levonorgestrel (3) wherein, the 13β-ethyl-3-methoxygona-2,5(10)-diene-17-one, referred hereinafter as methoxydienone (1), is subjected to ethynylation by reaction with lithium acetylide in tetrahydrofuran to obtain 13β-ethyl-3-methoxy-17α-ethynyl-gona-2,5(10)-dien-17β-ol, referred hereinafter as dienol ether (2). The dienol ether (2) is hydrolyzed by treatment with concentrated HCl in a mixture of methanol and water at room temperature to obtain levonorgestrel, which is purified by recrystallization from ethyl acetate. The process is depicted in the synthetic scheme 1 provided below:

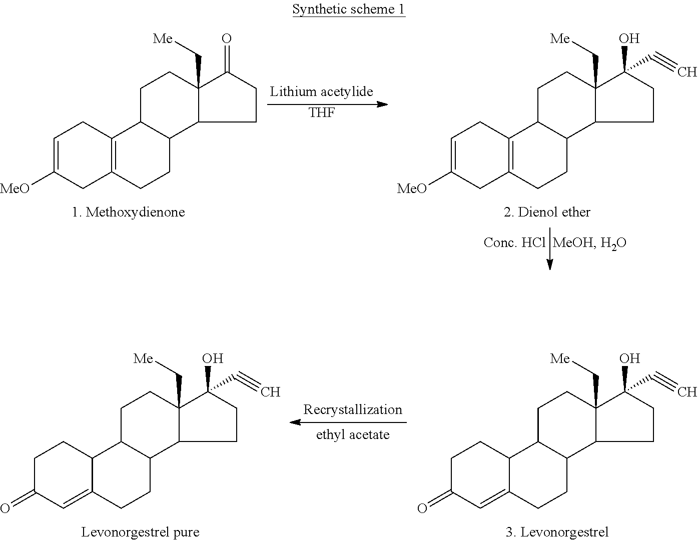 [SMILES: CC[C@]12CCC3C4=C(CCC3C1CCC2=O)CC(OC)=CC4 ; C#C[C@]1(O)CCC2C3CCC4=C(CC=C(OC)C4)C3CC[C@@]21CC ; C#C[C@]1(O)CCC2C3CCC4=CC(=O)CCC4C3CC[C@@]21CC ; C#C[C@]1(O)CCC2C3CCC4=CC(=O)CCC4C3CC[C@@]21CC ; "THF"]

The publications Rufer et al., Liebigs Ann Chem. (1967), 702, 141-8 and Helmut et. al; Helvetica Chimica Acta, 1985, 68, 1054-1068 further teaches a similar process for preparation of levonorgestrel (3) wherein methoxydienone (1) is subjected to ethynylation by reaction with lithium acetylide in tetrahydrofuran in presence of ethylene diamine to obtain dienol ether (2). Dienol ether (2) is hydrolyzed with HCl in methanol to obtain levonorgestrel (3), which is recrystallized from methanol.

The above-mentioned processes for preparation of levonorgestrel (3) lead to formation of an impurity, known as O-impurity, which is represented by structural formula provided below:

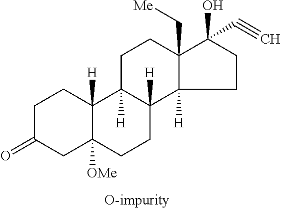

The O-impurity is described in European pharmacopoeia, Pharmaeuropa, 2010, vol. 22, No. 1, page 42-46. The O-impurity is probably formed due to addition of methanol across the C₅-C₁₀ double bond during hydrolysis of dienol ether (2).

The processes for preparation of levonorgestrel (3) described in the prior art suffer from following drawbacks:
(i) Low purity of levonorgestrel due to formation of O-impurity,
(ii) Low yield of levonorgestrel,
(iii) Repeated crystallizations are required to obtain pure levonorgestrel of pharmaceutical grade.

Thus, there is a need to develop a process which can produce highly pure levonorgestrel (3), which is free from O-impurity, in good yield.

### SUMMARY OF THE INVENTION

The present invention provides an improved process for preparation of levonorgestrel (3) comprising of ethynylation of methoxydienone (1) to obtain dienol ether (2) followed by hydrolysis of dienol ether (2) with an acid in aprotic solvent. The present invention also provides a method for purification of crude levonorgestrel (3).

### DESCRIPTION OF THE INVENTION

The present invention provides an improved process for preparation of levonorgestrel (3) comprising the steps of:
(i) ethynylating methoxydienone (1) to obtain dienol ether (2);
(ii) hydrolyzing dienol ether (2) with an acid in aprotic solvent to obtain levonorgestrel (3); and
(iii) optionally recrystallizing levonorgestrel (3) from a suitable solvent or mixture of solvents.

The process of present invention is depicted in synthetic scheme 2 provided below:

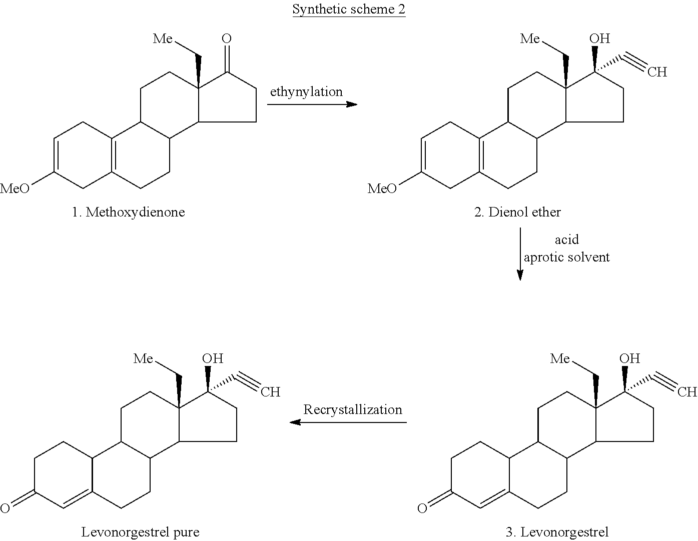 [SMILES: CC[C@]12CCC3C4=C(CCC3C1CCC2=O)CC(OC)=CC4 ; C#C[C@]1(O)CCC2C3CCC4=C(CC=C(OC)C4)C3CC[C@@]21CC ; C#C[C@]1(O)CCC2C3CCC4=CC(=O)CCC4C3CC[C@@]21CC ; C#C[C@]1(O)CCC2C3CCC4=CC(=O)CCC4C3CC[C@@]21CC]

The starting material, methoxydienone (1) can be prepared by methods known in the prior art documents: GB 1,010,053; GB 1,180,584; Helmut et. al., Helvetica Chimica Acta (1985), 68(4), 1054-68; Rufer et al., Liebigs Ann. Chem. (1967), 702, 141-8; and Quinkert et. al; Helvetica Chimica Acta (1995), 78(5), 1345-91.

In one aspect of the present invention ethynylation of methoxydienone (1) is carried out with an ethynylating agent in presence of a strong base in an organic solvent to obtain dienol ether (2).

The ethynylating agent is selected from the group consisting of acetylene or an alkali metal acetylide such as lithium acetylide, potassium acetylide or the like. Most preferably dry acetylene gas is employed as an ethynylating agent.

The base used for ethynylation reaction is selected from alkali metal alcoholates of tertiary alcohols such as potassium ter-butoxide, sodium ter-butoxide sodium ter-amylate, sodium ter-pentylate, potassium ter-amylate or the like in absence of any tertiary alcohol; organic amines such as ethylene diamine or liquid ammonia. More preferably alkali metal alcoholates are employed, most preferably potassium ter-butoxide.

The molar ratio of base with respect to methoxydienone (1) is in the range of 0.1 to 10 molar equivalents, more preferably 1 to 5 molar equivalents, most preferably 3 molar equivalents.

The solvent employed for ethynylation is selected from ethers such as dioxane, tetrahydrofuran, glycodimethyl ether, diethyl ether, diisopropyl ether; aromatic hydrocarbons such as benzene, toluene, xylenes or the like; polar aprotic solvents such as dimethyl formamide, N-methyl acetamide, N,N-dimethyl acetamide, dimethyl sulfoxide or any mixtures thereof.

The ratio of solvent employed for the ethynylation with respect to the methoxydienone (1) is 1 to 30 volumes, more preferably 5 to 15 volumes, most preferably 8-10 volumes.

Ethynylation is carried out under anhydrous conditions preferably at temperature of −25° C. to 40° C., more preferably at −10° C. to 0° C. The reaction is carried out preferably for 0.5 to 10 hours, more preferably for 3 to 6 hours.

The prior art method describes hydrolysis of dienol ether (2) with hydrochloric acid in methanol as a solvent. This method provides levonorgestrel containing O-impurity up to 0.29%. Surprisingly, the inventors of the present invention found that the content of O-impurity is below detection limit when hydrolysis of dienol ether (2) was carried out with an acid using aprotic solvent.

In another embodiment of the present invention, the dienol ether (2) obtained in the step (i) is hydrolyzed with an acid in an aprotic solvent to obtain levonorgestrel (3).

The acid employed for hydrolysis of dienol ether (2) is selected from mineral acids such as hydrochloric acid, sulfuric acid, phosphoric acid, nitric acid, perchloric acid; organic acids such as p-toluene sulfonic acid, methane sulfonic acid, acetic acid, formic acid and the like. More preferably a mineral acid is employed, most preferably concentrated sulfuric acid.

The molar ratio of acid with respect to dienol ether (2) is in the range of 0.5 to 10 molar equivalents, more preferably 3 to 7 molar equivalents, most preferably 5.5 molar equivalents.

Hydrolysis of dienol ether (2) is carried out in aprotic solvent selected from ketones such as acetone, ethylmethyl ketone, diethyl ketone, methylisobutyl ketone; ethers such as dioxane, tetrahydrofuran, glycodimethyl ether, diethyl ether, diisopropyl ether; aromatic hydrocarbons such as benzene, toluene, xylenes; amides such as dimethyl formamide, N-methyl acetamide, N,N-dimethyl acetamide; lower aliphatic esters such as ethyl acetate, methyl acetate, isopropyl acetate; halogenated hydrocarbons such as dichloromethane, chloroform, dichloroethane; dimethyl sulfoxide, acetonitrile or any mixtures thereof.

More preferably the reaction is carried out in a cyclic ether solvent, most preferably in tetrahydrofuran.

The ratio of solvent with respect to the dienol ether (2) is 1 to 20 volumes, more preferably 10 to 15 volumes, most preferably 12 volumes of solvent is employed for the hydrolysis.

The hydrolysis of dienol ether (2) is carried out at a temperature of 25-180° C. more preferably at a temperature range of 40-100° C., most preferably at 60-70° C. The reaction is carried out preferably for 0.5 to 10 hours, most preferably for 2-3 hours.

In another embodiment, the present invention also provides a process for purification of levonorgestrel (3) containing O-impurity by treatment with mineral acid in an aprotic solvent.

The aprotic solvents and mineral acids are same as that employed for hydrolysis of dienol ether (2) in step (ii), which are described above.

The ratio of solvent with respect to the levonorgestrel (3) is 1 to 20 volumes, more preferably 10 to 15 volumes, most preferably 12 volumes of solvent are employed.

The process of purification is carried out a temperature of 20-150° C. more preferably in a temperature range of 30-90° C., most preferably at 60-70° C. The mixture is stirred for 0.5 to 10 hours, preferably for 2-3 hours.

In another aspect of the present invention, levonorgestrel (3) obtained in step (ii) is further optionally purified by recrystallization from a suitable solvent or mixture of solvents.

The solvent employed for recrystallization is selected from lower alcohols such as methanol, ethanol, n-propanol, isopropanol; ketones such as acetone, ethylmethyl ketone, diethyl ketone, methylisobutyl ketone; ethers such as dioxane, tetrahydrofuran, glycodimethyl ether, diethyl ether, diisopropyl ether; aromatic hydrocarbons such as benzene, toluene, xylenes or the like; polar aprotic solvents such as dimethyl formamide, N-methyl acetamide, N,N-dimethyl acetamide, dimethyl sulfoxide, acetonitrile; lower aliphatic esters such as ethyl acetate, methyl acetate, isopropyl acetate; halogenated hydrocarbons such as dichloromethane, chloroform, dichloroethane or any mixtures thereof. More preferably recrystallization is carried out in a mixture of water and a polar organic solvent, most preferably in a mixture of dimethyl formamide-water; methanol-water.

The ratio of water:polar organic solvent in the mixture is in the range from 10:90 to 1:99 (v/v), preferably 5:95 to 1:99 (v/v). Most preferred solvent ratio of water:methanol is 1:99 (v/v).

Recrystallization is carried at reflux temperature of the solvent employed and the solution of levonorgestrel (3) is stirred at reflux temperature preferably for 0.1 to 10 hours, more preferably for 0.5 to 1 hour.

The process of present invention not only eradicates formation of O-impurity and produces highly pure levonorgestrel in good yield but also obviates the need to carry out repeated crystallizations of the product.

The invention is further illustrated with reference to the following examples. It is apparent to those skilled in the art that many modifications, both to materials and methods, may be practiced without departing from scope of the invention.

The HPLC method employed for analysis of levonorgestrel is as follows:

Column: Symmetry Shield RPB, (4.6×250 mm), 5 μm
Eluant: mobile phase A=60:40 mixture of water and acetonitrile, mobile phase B=HPLC grade acetonitrile.
Gradient: 0 to 50 minutes, concentration of mobile phase B=0 to 80%
Flow rate: 0.7 mL/min
Detector wavelength: 200 nm
Injection volume: 50 μL
Column temperature: 30° C.
Diluent: 30:70 mixture of water and acetonitrile

### Example 1

### Preparation of Dienol Ether (2)

22.3 g of potassium ter-butoxide was charged in 100 ml of tetrahydrofuran (moisture content NMT 0.1%) under nitrogen atmosphere at −10° C. to 15° C. and flushed with 40 ml of tetrahydrofuran. The mixture was stirred and acetylene gas was purged in to the mixture for 1 hour. A slurry of 20 g of methoxydienone (1) in 60 ml of tetrahydrofuran was charged in the reaction mixture and stirred with continuous purging of acetylene gas for 3 hours at −10° C. to 0° C. 400 ml of DM water was added and pH was adjusted to 5 to 6 by addition of aqueous H₂SO₄ solution. 200 ml of dichloromethane was added, the mixture was stirred and layers were separated. The aqueous layer was extracted with 200 ml of dichloromethane, layers were separated and organic layers were combined and washed with water. The organic layer was distilled to obtain solid residue. The residue was stirred in 60 ml of methanol, filtered and washed with methanol. The wet cake obtained was dried under vacuum 45-50° C. to obtain white solid.

### Yield=16.3 gm (75%)

### HPLC Purity=85.68%

### Example 2

### Preparation of Levonorgestrel (3)

A solution of 10 g of dienol ether (2) in 120 ml of tetrahydrofuran was treated with 45 ml of 20% v/v sulphuric acid solution. The mixture was stirred at 65-68° C. for 1-2 hours. After completion of reaction, the reaction mass was cooled to 20-30° C. and 50 ml of DM water was added. The solid precipitated was filtered and slurried in 50 ml of water. 23% aqueous ammonia solution was added till pH of the mixture was 7 to 8. The mixture was stirred, filtered and the wet cake was washed with water and suck dried under vacuum to obtain white solid.

### Yield=8.1 gm (84.6%)

### HPLC Purity: Levonorgestrel=98.3%

O-impurity=BDL

### Example 3

Recrystallization of Levonorgestrel (3)

A mixture of 8 g of levonorgestrel (3) in 360 ml of methanol was refluxed at 65° C. to get clear solution. 10 g of activated carbon was added and stirred. The hot the solution was filtered and the filter bed was washed with 8 ml of methanol. The clear filtrate was distilled under vacuum till 10 volumes of the filtrate remained. The slurry obtained was cooled to 20-30° C., stirred, filtered and the crystalline white solid obtained was dried under vacuum.

### Yield=6.8 gm (85%)

### HPLC Purity=99.52%

### Example 4

### Recrystallization of Levonorgestrel (3)

40 g of levonorgestrel (3) was dissolved in 320 ml of dimethyl formamide at 58-62° C. to get clear solution. Activated carbon was added to the hot solution, stirred, filtered and washed with 20 ml of dimethyl formamide. 170 ml of DM water was added to the filtrate at 58 to 62° C. to crystallize the product. The mixture was cooled to 20-30° C., stirred and filtered. The crystalline white solid obtained was dried under vacuum.

### Yield=34.5 gm (86%)

### HPLC Purity=99.46%

### Comparative Example 5

### Preparation of Levonorgestrel (3) by Prior Art Method

150 g of dienol ether (2) was charged in 2250 ml of methanol and the mixture was heated at 55-60° C. for 1 hour. 510 ml of concentrated HCl was added and the mixture was stirred at 55-60° C. for 3 hours. The reaction was cooled to 10-15° C. and stirred for 4 hours. The solid obtained was filtered, washed with DM water followed by washing with 300 ml of 23% aqueous ammonia solution and again with DM water and methanol. The solid obtained was dried under vacuum.

### Yield=121.5 g (84.6%)

HPLC purity: Levonorgestrel=98.65%
O-impurity content=0.29%

### Example 6

Purification of Crude Levonorgestrel (3) Containing O-Impurity:

To a solution of 10 g of crude levonorgestrel (3) containing 0.29% of O-impurity in 120 ml of tetrahydrofuran, 30 ml of 20% v/v sulphuric acid solution was added and the mixture was stirred at 60-68° C. for 1 hour. The reaction mass was cooled to 20-30° C., stirred for 2-3 hours and 50 ml of DM water was added. The mixture was stirred, filtered and washed with 20 ml of DM water. The wet cake was slurried in 100 ml of water and 23% aqueous ammonia solution was added till pH of the mixture was 7 to 8. The solid was filtered, washed with water and dried under vacuum to afford white crystalline solid.

### Yield=8.5 gm (85%)

### HPLC Purity: Levonorgestrel=98.8%

O-impurity=BDL

1. A process for preparation of levonorgestrel (3) 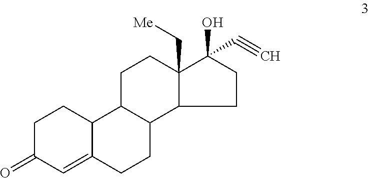 [SMILES: C#C[C@]1(O)CCC2C3CCC4=CC(=O)CCC4C3CC[C@@]21CC]
comprising the steps of:
(i) ethynylating methoxydienone (1) to obtain dienol ether (2); 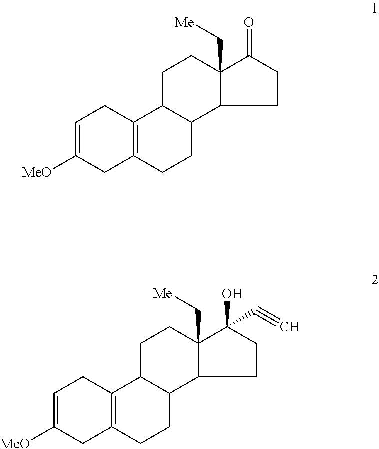 [SMILES: CC[C@]12CCC3C4=C(CCC3C1CCC2=O)CC(OC)=CC4 ; C#C[C@]1(O)CCC2C3CCC4=C(CC=C(OC)C4)C3CC[C@@]21CC]
(ii) hydrolyzing dienol ether (2) with an acid in aprotic solvent to obtain levonorgestrel (3);
and
(iii) optionally recrystallizing levonorgestrel (3) from a suitable solvent or mixture of solvents.

2. A process according to claim 1, wherein the aprotic solvent is selected from ketones such as acetone, ethylmethyl ketone, diethyl ketone, methylisobutyl ketone; ethers such as dioxane, tetrahydrofuran, glycodimethyl ether, diethyl ether, diisopropyl ether; aromatic hydrocarbons such as benzene, toluene, xylenes; amides such as dimethyl formamide, N-methyl acetamide, N,N-dimethyl acetamide; lower aliphatic esters such as ethyl acetate, methyl acetate, isopropyl acetate; halogenated hydrocarbons such as dichloromethane, chloroform, dichloroethane; dimethyl sulfoxide, acetonitrile or any mixtures thereof.

3. A process according to claim 2, wherein the aprotic solvent is tetrahydrofuran.

4. A process according to claim 1, wherein the acid is selected from mineral acids such as hydrochloric acid, sulfuric acid, phosphoric acid, nitric acid, perchloric acid; organic acids such as p-toluene sulfonic acid, methane sulfonic acid, acetic acid, formic acid.

5. A process according to claim 4, wherein the acid is sulfuric acid.

6. A process according to claim 1, wherein molar ratio of acid with respect to dienol ether (2) is in the range of 0.5 to 10 molar equivalents.

7. A process for purification of levonorgestrel (3) containing O-impurity, wherein levonorgestrel is treated with mineral acid in aprotic solvent.

8. A process according to claim 7, wherein the mineral acid is selected from hydrochloric acid, sulfuric acid, phosphoric acid, nitric acid and perchloric acid.

9. A process according to claim 8, wherein the acid is sulfuric acid.

10. A process according to claim 7, wherein the aprotic solvent is selected from ketones such as acetone, ethylmethyl ketone, diethyl ketone, methylisobutyl ketone; ethers such as dioxane, tetrahydrofuran, glycodimethyl ether, diethyl ether, diisopropyl ether; aromatic hydrocarbons such as benzene, toluene, xylenes; amides such as dimethyl formamide, N-methyl acetamide, N,N-dimethyl acetamide; lower aliphatic esters such as ethyl acetate, methyl acetate, isopropyl acetate; halogenated hydrocarbons such as dichloromethane, chloroform, dichloroethane; dimethyl sulfoxide, acetonitrile or any mixtures thereof.

11. A process according to claim 10, wherein the solvent is tetrahydrofuran.
